(19) United States
(12) Patent Application Publication
(10) Pub. No.: US 2010/0178204 A1
(43) Pub. Date: Jul. 15, 2010

(54) Automated Self-Contained Liquid Handling and Detection System Device

(75) Inventors: Ray Yin, Newark, DE (US); Ankuo Wang, Santa Ana, CA (US); Songtao Li, Arcadia, CA (US)

Correspondence Address: MDIP LLC, POST OFFICE BOX 2630, MONTGOMERY VILLAGE, MD, 20886-2630, US

(73) Assignees: ANP Technologies, Newark, DE (US); Cogent Systems, Inc., South Pasadena, CA (US)

(21) Appl. No.: 12/377,125

(22) PCT Filed: Aug. 21, 2007
(86) PCT No.: PCT/US07/76426

§ 371 Date: Mar. 4, 2009

Related U.S. Application Data
(60) Provisional application No. 60/823,034, filed on Aug. 21, 2006.

(51) Int. Cl. G01N 35/00; G01N 35/10; G01N 33/563; G01N 33/53; G01N 33/487

(52) U.S. Cl. 422/63; 422/81

(57) ABSTRACT

An integrated device which combines automated liquid handling, rapid planar binding assay format, reservoir of assay devices, automated package opening device and automated reader for obtaining assay results, with an optimized means for data analysis, storage and management, is described.

### FIELD OF THE INVENTION

The present invention concerns the use of a self-contained, automated liquid sampling and handling, and reaction detection system device for analytical assay applications related to use, for example, in agriculture, environmental studies, diagnostics, drug monitoring, drug target screening, lead optimization and so on. A suitable assay format that can be exploited by the device of interest is the lateral flow immunoassay.

### BACKGROUND OF THE INVENTION

Although a large number of biological assays including immunoassays and nucleic acid-based assays have been automated for various applications, analytical tests such as immunochromatographic (or lateral flow) assays have been primarily used in non-automated, rapid manual/point of care applications. Lateral flow assays offer the convenience of simplicity and rapid time to result. However, automation can be complicated by the size of the lateral flow assay device and the instability of reagents found on the stored assay device under high humidity conditions. Ambient moisture can rapidly destroy the dried reagents on the lateral flow device, thus degrading the performance of such assays. Heat also can further accelerate the reagent decay process.

For those reasons, lateral flow assays and other analytical test technologies that are susceptible to such environmental challenges are difficult to use in field conditions where humidity cannot be controlled. Those assays are also difficult to automate due to the packaging requirements. To protect the lateral flow assay devices from humidity, the lateral flow devices generally have to be individually packaged in sealed polymer or metal foil-polymer laminates that are efficient moisture barriers. Bulk packaging of those tests in a single foil-lined container is not effective since once the seal is broken, the remaining tests will be exposed to ambient humidity. The difficulty in manipulating, opening and disposing of the individual protective packaging presents a daunting challenge to automating the use of such tests using a single, compact and multifunctional handling and detecting device.

### SUMMARY OF THE INVENTION

The present invention is directed to an automatic, self-contained liquid sample handling and detection system device that comprises an automated liquid handling means, an automated assay device package opening means and a reader, that is, a detecting means to recognize reporter molecules or signals therefrom on the assay device, optionally, with automated data analysis means, such as, software, for use, for example, with both single target and multiplexed assays, such as, lateral flow assays, such as, lateral flow immunoassays. Such lateral flow assays can be packaged in a sealed roll format, where each lateral flow assay device is individually, removably sealed, for example, with a flexible, membrane-type sealant, to avoid exposure of the ready to use lateral flow devices to the environment, and particularly moisture. Immediately before testing, the assay device is exposed, for example, a removable film attached to and over the individual assay device is removed one at a time by an automated mechanical means so that only one assay device is exposed to the ambient, outside environment at a time. The assay device can be transported by a movable platform, such as a belt. The liquid sample then is introduced to each exposed, uncovered assay device by an intelligent sampling and applying means to enable assay operation, such as, lateral flow of the fluid phase, immune or binding reaction and signal development. The remainder of the assay devices, such as, lateral flow assay devices, remain in the original, sealed packaging until needed.

On completion of the reaction and development of a detectable signal, as controlled by a timing means and executed by the timed movement of the assay device, such as, a lateral flow device, on the movable platform, that is, the platform moves at a speed, which following application of the sample on, onto or to the assay device, ensures, in the case of a lateral flow assay device, fluid movement across the lateral flow assay device, the reaction to occur and development of a detectable signal. The lateral flow assay device then is moved to enable a detection means to scan and to detect any signal molecules present at the detection site of the assay device to indicate, for example, a positive reaction, if the reaction mechanism is dependent on direct assay where the strength of signal correlates directly or positively with the amount of analyte, or a negative reaction if the reaction mechanism is in the form of a competition assay where the strength of signal correlates indirectly or negatively with the amount of analyte. The “reader” device, the detection means, recognizes the signal, scans and digitizes the signal, which data then is analyzed and processed to yield a diagnostic metric and/or is stored in a data storage means, which can be local in the system device of interest or can be remote, wherein the data is communicated from the system device to a data storage means by suitable data communication means, such as a removable and portable data storage means, a wireless means and so on. The data can be processed and analyzed using suitable processing means, such as, software, installed in either the system device or in a remote processing means, such as, a computer.

After data analysis is completed (often in seconds), the used assay device or ticket is further transported, in the case of the sealed roll format, to the waste roll located at the opposite end of the system device by the movable platform means therein. Accordingly, the next assay ticket will be opened and run in the same sequence when needed as described above.

The present automatic detection system device will be useful for biodetection, medical and food diagnostics, and pharmaceutical discovery related applications, for example.

### BRIEF DESCRIPTION OF THE FIGURES

The following description of the figures and the respective drawings are non-limiting examples that depict various embodiments that exemplify the present invention.

FIG. 1 depicts a side view of an embodiment of an automated, self-contained liquid handling and detection system device. The individual lateral flow immunoassay devices are identified as tickets.

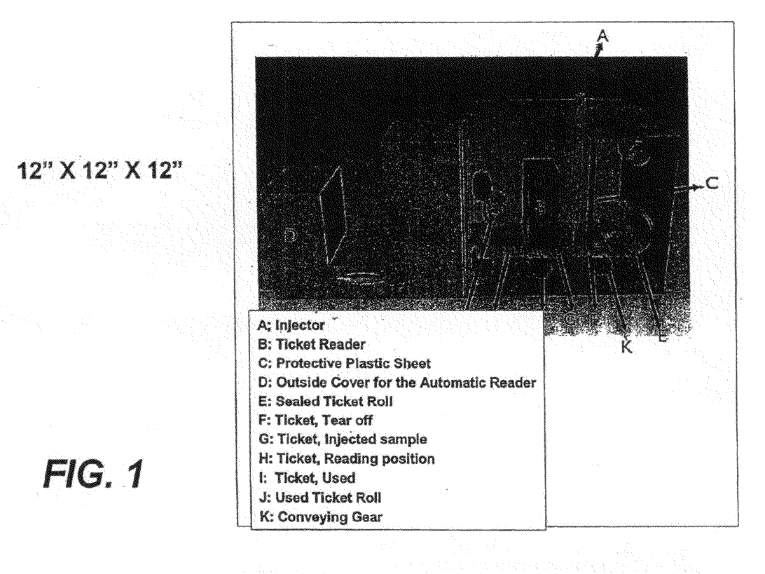

FIG. 2 depicts a side view of another embodiment of the automated, self-contained liquid handling and detection system device with detailed assay ticket assembly, liquid dispenser, assay reader and continuous package peeling device.

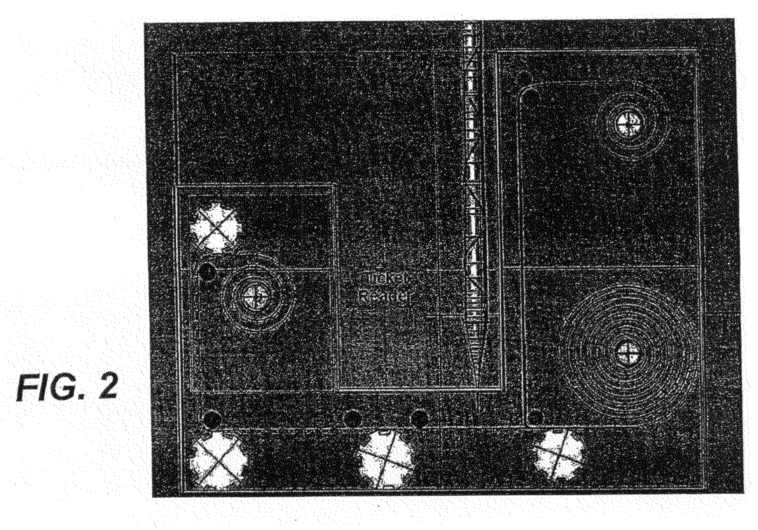

FIG. 3 depicts a superior view of an embodiment of a representative assay ticket assembly with sample addition wells situated toward the top of the figure, the injector (b) is situated over a sample addition well of a ticket.

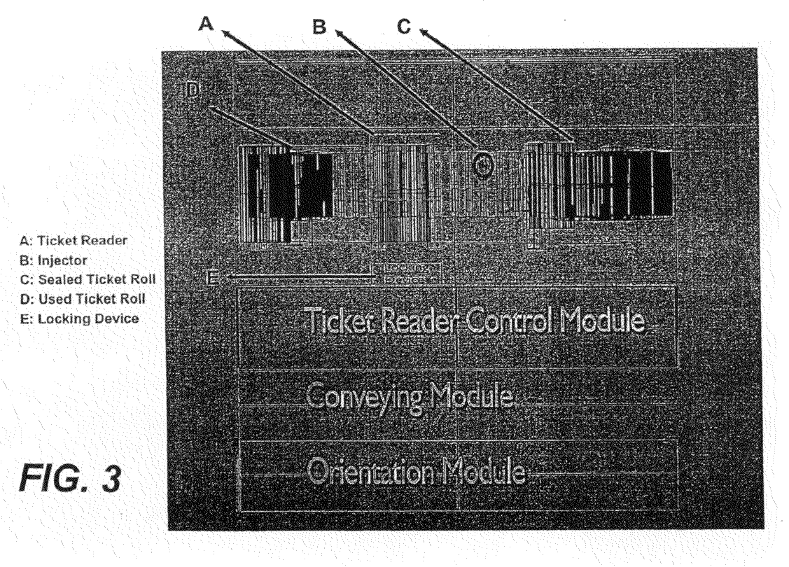

FIG. 4 depicts multiplexed lateral flow immunoassay assays in both line and dot format.

### DETAILED DESCRIPTION OF THE INVENTION

The concepts herein disclosed are solutions to the aforementioned problems. The concepts allow each assay device or ticket to remain individually packaged in a sealed moisture barrier that either is opened only immediately before an assay is to be performed or is one of a plurality of devices maintained in a sealed container means, wherein individual assay devices are removed in a manner that retains the low moisture environment for the remainder of the unused assay devices in the container means. A container means can be a disposable housing made of, for example, a plastic material, having a suitable configuration and shape to contain the assay devices of interest and which can interface with the system device of interest. Presented are means for the storage of ready-to-use assay devices in a compact manner, an uncomplicated means to retrieve an individual packaged assay device, to remove the packaging and disposing of the packaging material, to deliver the assay device to a work station where the assay result is determined and to recover and sequester used assay devices for future disposal. As an integrated whole, the concepts enable an automated assay device system device that is usable with any sort of planar assay device requiring protection from humidity. The overall system device can be integrated with a sample storage means and/or a sample delivery means. The sample storage means can be one which contains a single or a plurality of samples, such as, a carousel, which may be configured with a means to position the sample to be tested to the liquid handling means of the system device. The sample storage means can be configured to maintain the samples in a state which preserves the sample, such as, at reduced temperatures. The data obtained by the detecting means from the individual assay devices can be digitized and stored. The data obtained also can include a scan of the assay result portion of the assay device or a digital representation of the reacted assay device. The data can be stored, interpreted or analyzed by an integrated or remote processing means.

For example, individual assay devices (also known as “tickets”) can be placed in pouches or voids formed by a sealable means, such as, two ribbons or membranes of sealable flexible material, one ribbon of which can serve as a carrier and transport means for the tickets. The other ribbon thus can serve as the means to seal each assay device in a space or void formed by the sealing of the two ribbons, the material being sealable to or over the superior surface of the ticket, that seal being one which provides voids for encasing an assay device of interest in an environmental barrier means to protect the ticket contents from the environment.

In one embodiment, the two ribbons can be sealed to each other just outside the perimeter of the ticket. The seal is on all four sides of the ticket, forming an air-tight and moisture-proof pouch or void for the ticket. As the lateral flow devices generally have a rectangular presentation with longer and shorter axes, normally, the tickets are placed on the inferior ribbon of sealing material with the long axis of the ticket oriented along the short axis of the sealing material, which generally is a roll or strip of material with the long axis along the length of the roll or strip. Thus, the shorter axis of the ticket runs along the longer axis of the sealing material, essentially along the length of the roll. The two ribbons are sealed at specified intervals across essentially the length of the shorter axis of the sealing material, the ribbon, around the ticket to form the sealed pouch or void, with intervening open spaces between adjacent tickets at specified intervals, spacers. The spacers can be of a length that separates the assay devices suitably to enable access and use of a single assay device by the system device without exposing unneeded assay devices to the ambient environment.

A sealing means attaches the superior sealing material film, onto and over the ticket, with the lower sealing material, for example, to contain the tickets individually. The sealing means can be, for example, heat, an embossed pressing, a chemical, a sealant, such as a glue, electric current and so on.

One end of the dual-layered ribbon having individually contained assay devices therein, referred to as the starter end, can be left unsealed, creating a bottom leaf and a top leaf. In one embodiment, each leaf is fed onto a collecting means for storing the separated leafs or membranes. An example of a suitable collecting means is an uptake spool. The ribbon of sealed devices can be coiled and the coil can be placed on or can include a spindle which resides at or as a “feeder” spool. One uptake spool is for the separated and removed superior sealing film, which membrane separation process exposes the ticket. The other uptake spool is for collecting the other, inferior sealing film, which can be the transporting film carrying the tickets throughout in the system device. As each spool pulls the respective leaf a specified distance, the sealed halves of the ribbon are separated. In that manner, a single sealed pouch containing an assay device is opened, releasing the assay device for immediate use while keeping the remainder of the assay devices in the ribbon of pouches, unopened.

In one possible design, as shown in the Figures, the assay device will stay with the bottom leaf, which delivers the assay device by a transporting means, such as a stepping or moving belt, to a work station, which together or separately includes a liquid handling means for sample injection and a detecting means where the assay results are measured, digitized, communicated, stored and so on. After the assay is concluded, the assay device is transported on the lower leaf to an uptake reel that holds used assay devices. Hence, in such an embodiment, the assay device is affixed to the inferior leaf. In an alternative embodiment, the assay device is removably placed on the inferior leaf, and after use, is removed from the inferior leaf and discarded into a used assay device collection means. While the assay is being performed, the remaining assay devices remain sealed and protected within their sealed pouches. When all assay devices within a spool are used, the uptake reels containing the used assay devices and the sealing, superior film are removed and discarded and a new coil of sealed unused assay devices is put in place in the feeder spool.

In an alternative embodiment, a plurality of tickets are placed in sealed packaging or container means that serve essentially as cassettes containing plural assay devices in a configuration that enables the system device to extract individual tickets as needed. Such a container means provides a portable contained set of assay devices of varying number. The container means is configured to removably engage suitable integrated holding means, such as a bracket or a housing for accepting the assay device container means, in the system device which enables the cassette to be situated in the system device, and as tickets are needed, the container means allows for individual removal of assay devices in a manner that, optionally, retains the low humidity, dry environment within the container means to stabilize and to extend the shelf life of the as yet to be used assay devices. Hence, the container means can comprise an egress means that enables removal of single assay devices therefrom in a manner that maintains the state of the container means interior environment. Thus, the container means can comprise a baffled, layered, pleated, feathered, multi-leafed, diaphragm, collapsible means, such as, a rubbered stopper, a gelled stopper, and so on, means that enables removal of an individual assay device while allowing a minimum of no air entry into the container means during the removal process. The container means can contain an interior under positive pressure, such as, infused with nitrogen gas or other inert gas, to ensure that as an assay device is removed from the container means, the propensity is for any gas flow across the container means egress to be from inside to outside rather than from outside to inside. The container means can contain a means to position the unused tickets within in a manner that enables the system device to extract all individual tickets despite orientation of the container means within the system device. For example, the tickets can be stacked within the container means under tension, such as, by a spring means or tensioning means to enable positioning of a ticket at the site of egress in and from the container means. Examples of packaging or container means containing a plurality of removable objects, wherein the objects are under a tensioning means to enable orderly and complete removal of all objects from said packaging or container means include, for example, containers containing single edge razor blades and magazines containing armament. Alternatively, the packaging or container means need not be configured to maintain and to retain a low humidity environment within on removal of the first and subsequent assay devices, for example, if the interior of the system device is a contained low humidity environment or the assay devices are used at a steady and fast pace or rate.

Other means of designing, forming and storing prior to use individual, sealed tickets can be contemplated and used in the practice of the instant invention.

The assembly or system device comprising the various elements taught herein can be contained in a housing that encompasses most, if not all, of the various components of the system device of interest, that optionally, can be integrated within a larger system device, for example, containing continuous air or water samplers and handlers, such as, for sample disposal. The housing can include a protective casing composed of, for example, a plastic or metallic material, as well as areas composed of a transparent material to enable the user to view interior portions of the system device. The housing also can include various data manipulation means, such as a data entry means, such as a keyboard, and a system device control means to enable the user to interface with any data processing means contained within the system device, to direct the flow of data from said system device, operate portions thereof and so on, as well as to operate, manipulate, debug and so on one or more components of the system device. The integrated system device becomes adaptable for portable field-use as a stand-alone system device that can be employed, for example, for automated surveillance. When integrated with sample handling systems for biological samples, such as blood, serum, plasma, urine, and cell or tissue preparations, the designs of interest enable and provide a clinical laboratory or point-of-care automated diagnostic system device. The housing also can include a power or energy source and/or conduit means to enable operation of the various mechanized and processing means portions thereof, including both AC and DC sources, including, for example, storage cells, batteries, fuel cells, solar panels, wire means for connection to power or energy sources and so on.

The transporting means within the reader system device can be in the form of a flexible belt material that is configured to engage a belt movement means, such as a step motor and so on, which enables a directional movement of the belt, and thus of the tickets. The belt can be made from metallic sources, such as braided metal cloth, linked metal rings and son on, a cloth material, a polymer material and so on.

Alternatively, the lower film or sealing material can be of a heavier character with, for example, a lower level of elasticity along its longer axis, wherein the system device contains transportation means to advance movement of the lower sealing film carrying the ticket along the length of the strip. For example, the lower film can have along the length of the film, at each edge, a regular series of notches or holes which engage toothed gears, which are sited and turnable in a manner where the teeth engage the holes in the lower membrane or leaf and advance the membrane or leaf by a coordinated movement, similar to the configuration found on silver nitrate-based photographic films (35 mm film) and their means of advancement of one frame to another by a series of gears which engage a regular series of holes provided at each edge along the length of the photographic film in a camera.

The transportation means can be controlled by suitable devices to enable movement of the tickets in a coordinated fashion to provide an adequate time for the various steps, such as, the assay reaction, to occur once the sample is added to the ticket. Thus, a data processing means with appropriate effector means can be used to operate and to coordinate the actions of the various components of the liquid handling and detection components of a system device of interest. In another embodiment, advancement of assay devices can be controlled manually by the user, for example, including a controlling means to advance the transporting means to expose an assay device for use, for example. Thus, for example, a foot pedal, a switch, a switching means, an activating means and so on can be integrated into and with the system device that enables the user to control operation of the system device. In such an embodiment, the assay device can be transferred to another, second transporting means which communicates the exposed assay device to the various work stations of the system device. The remainder of the unused assay devices remain on the feeder spool, on the first transporting means.

The liquid handling means can be multifunctional and its movements and actions governed by a computer processing means. The liquid handling means can have a sample withdrawal or acquisition means, such as, a vacuum or suction function, to enable drawing samples, buffers and the like, as well as an expelling or applying means, such as, a pressure means to expel contents. The sample withdrawing and expelling functions can be obtained as known in the art using hydraulic means, diaphragms, pistons and the like to provide a positive displacement or negative displacement means to handle and move liquids. The liquid handling means can contain multiple fluid conduits within to carry plural samples simultaneously, for example, or to deliver plural liquids simultaneously or different liquids sequentially, for example, under control of a processing means. The sample conduit can be single use and replaceable, or the conduit can be flushed with a suitable wash liquid, being expelled into a suitable waste liquid container or other disposing means. The liquid handling means also can be configured to be movable in plural directions, at the least, in an x and y direction, as well as up and down, relative to the assay device surface to enable flexible movement of the liquid handling means within the system device. The liquid dispensing means can be appended with a supporting means, a bracket, a guide and so on to provide for orderly and accurate application of liquids to the assay device. The method and means for making such robotic devices is known in the art, and a number of different embodiments can be used in the practice of the instant invention.

The sample is a liquid sample, which can be a body fluid, used neat or diluted as needed with a suitable diluent, solids dissolved or suspended in a suitable diluent, which dissolved or suspended sample may be separated or filtered to remove particulates prior to assay device application, can be any liquid, used neat or diluted as needed using a suitable diluent and so on. Examples of body fluids include urine, serum, tissue extracts, spent culture medium and so on.

The ticket reader system device can contain a means for detecting the reporter molecule or signal emitted or released therefrom. The means can be, for example, a photodetector, a colorimeter, a fluorimeter, a spectrophotometer, any type of automated sensor which can discern the reporter molecule used.

The detected signal can be digitized for data processing and analysis, as well as for storage. The system device detecting means can be mobile to enable the ticket to be read at a defined period of time following reaction, independent of the transporting means. The detecting means can obtain a scan and/or data from the ticket. The image or data are digitized for use by a processing means. There, the data are processed to yield a readout, such as a qualitative “yes/no,” “positive/negative,” “reaction/no reaction” and so on determination of whether a reaction occurs, with a user determined threshold of what constitutes a positive or negative result, or a quantitative presentation of the assay results to yield alternative information for analysis.

The quantitative result can range from the absolute data obtained from the assay to one which contains statistical parameters to one which includes an assemblage of data from a number of samples to provide a sample population of which the assay result obtained is a member, the population may include population statistics, such as a graph or plot of data points, to provide a depiction of certain population statistical parameters, along with confidence limits, with the obtained test result highlighted in the population data. The nature of the display is a design choice.

Any binding assay format that is configured in a planar presentation that is configured to yield a detectable signal thereon can be used in a system device of interest. The binding assay, such as a receptor assay, one using an aptamer or one using an antibody, for example, can be in any format, such as direct or indirect, competition, sandwich and so on, as known in the art. The assay can contain intermediate binding molecules to compliment or assist with the detection of analyte, such as a receptor and ligand, such as avidin and biotin, which serve a subsidiary bridging function. The reaction is configured to occur on a planar surface, such as, a dipstick-type assay or a lateral flow assay.

Suitable means for providing a detectable signal are known in the art and reporter molecules, such as, light-emitting molecules, for example, fluors, chemiluminescent molecules and so on, colloidal particles including colloidal gold, colloidal silver and colloidal platinum, polymer or latex beads, inorganic particles, colored latex particles, particles containing fluorescent or colored materials, clay, ceramic, silicon-based or ceramic semiconductor particles, silicon or ceramic semiconductor chips, nanocrystals, quantum dots, which are inorganic nanoparticles (often less than 5 nm in diameter) capable of emitting different colors of light by controlling the composition and size of the material contained within the particle, up-converting phosphors, which are submicron ceramic microparticles that emit visible light on excitation with near-infrared light, enzymes, such as alkaline phosphatase and peroxidase, and so on. The joining of a desired reporter molecule to a binding reagent of interest can be effected using reagents and practicing methods known in the art.

WO 2005/051295, WO 2006/102484 and WO 2006/119160 are examples of references teaching the sort of assay devices that can be used in the automated liquid handling and detecting system device of interest.

When the system device has a data processing means, the assay result can be presented on the system device by a display means. The display means can take a number of formats and presentations, such as a light that is configured to illuminate when a positive or negative result is obtained, there can be a more detailed display means, such as a diode array, or a liquid crystal display, or the system device can include a printing means whereby the result is printed in a suitable user selected form and format. The system device can have a processing means to effect operations of the system device. The system device processing means can be included with or integrated with a data processing means in a central processing means.

The system device can serve merely a data acquisition or collection function, in which case the data processing, analyzing and permanent storing functions are handled remotely from the system device. Any form of data storage means can be used, including, the use of data storage devices, such as diskettes, CD's, DVD's, magnetic tape, mass storage devices, such as flash drives, and so on, or can include a data communication means for transfer, which can be real time, of data from the system device to a remote site, using, for example, telephony communication means, such as wire, fiber, coaxial cable, electric power lines and so on, or using wireless means, using, for example, radio frequency (RF), infrared light, microwave, laser light, visible light, acoustic energy and so on, as known in the art.

To ensure coordination, and to provide an identification means, each ticket can be uniquely labeled, such as with a bar code, to provide a unique identifier as well as providing a means for the system device to track any one ticket, for example, to know when sample is applied so that an appropriate time passes, at which time the ticket detecting means is instructed to move the appropriate ticket bearing the appropriate identifier and to detect and read the signal, as well as coordinating the assay result with a particular sample. In the case of bar coding, the system device can include a number of suitable means for scanning and recognizing a bar code, for example, a sensing means can be placed to read the bar code of a ticket just prior to sample addition, and a plurality of additional sensing means can be included along the path of assay device movement to monitor the location of an assay device in the system device. Also, an opened assay device can be labeled, for example, with the time of opening or time of sample application, to supplement the processing means monitoring of assay device movement in the system device or to provide a physical labeling of the assay device for visual confirmation.

It will be appreciated that the particular embodiments described herein are merely exemplary and the invention can take a number of different formats and presentations that will enable the practice of the instant invention. The features of interest are to provide an automated means of conducting, for example, lateral flow immunoassays, wherein the individual assay devices are individually wrapped to enhance shelf life.

The invention now will be exemplified in the following non-limiting examples.

### Examples

Various color or fluorescent based assays can be utilized in the present automated detection system device. These assays may include, but are not limited to, lateral flow tests of various configurations, flow-through assay systems, dry-chemistry analytical tests, color change strips (such as glucose strips and occult blood tests), etc.

The following example demonstrate that one lateral flow assay design can be used in such an automated detection system device.

Gold-Ab conjugates are made as follows. To a 125 ml flask were added 60 ml of colloidal gold solution (20-80 nm in diameter as measured by TEM, O.D. 1.078 as measured by UV spectroscopy) (Frens et al., Nature Phys Sci 241:20-22, 1973). The pH of the solution was adjusted to 8-11 by addition of a 0.2 M potassium carbonate solution. To this solution were added 600 μl of conjugated antibody solution (O.D. 0.1-1.5 in sodium borate buffer) while stirring, followed by subsequent addition of 600 μl of bovine serum albumin (20% with sodium azide stabilizer). The mixture was stirred at 20° C. for 20-240 more minutes. The solution remained purple in color and some foaminess was observed. On completion, the stir bar was removed, and the reaction mixture was transferred to two 50 ml conical tubes. The material was centrifuged until very little color was observed in the supernatant. The supernatant was removed and 600 μl of 25 mM sodium borate buffer were added in each tube. The contents were mixed thoroughly and the two tubes of material were combined and characterized by UV-Vis.

The gold-modified branched polymer (MBP)-streptavidin conjugates were prepared in a similar manner. The gold-MBP-streptavidin-biotin-Ab conjugates were prepared through subsequent addition of biotinylated Ab to gold-MBP-streptavidin conjugates. Standard antibody biotinylation protocols can be found for example, in Bioconjugate Techniques (G. T. Hermanson, Academic Press, 1996). Other biologically active molecules, which can be used as reporters, such as horseradish peroxidase (HRP) or avidin and derivatives and analogs thereof can also be attached to gold in a similar manner. However, during the test, additional substrates have to be added to achieve signal enhancement.

An immunoassay device or “ticket” can consist of a strip of cellulose or other membrane in a membrane-retaining device, generally composed of an inert plastic, an adsorbent pad and a receiving pad at the ends of the membrane.

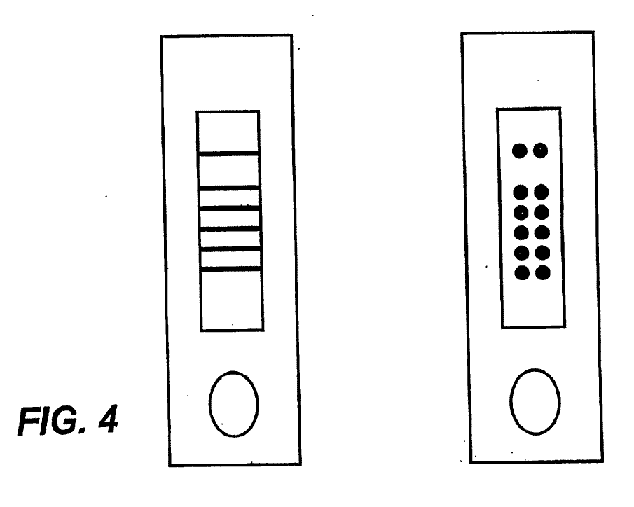

FIG. 4 illustrates two or many different formats of lateral flow-based immunoassay configurations. For example, the detection zones can be in the form a line or a spot. The immunoassay ticket can have a plastic cover forming a discrete sample application site, the oval, and a discrete test site. Such a cover also minimizes environmental exposure, provides structural support and retards evaporation when a sample is applied.

A number of different assays of varying formats were constructed and tested amongst similar such assays containing a conjugate of interest and also with assays not containing a conjugate of interest, for example, having a directly labeled detector molecule.

The assays containing a conjugate of interest were superior.

It will be apparent to one skilled in the art that various changes, alterations, and modifications of the present invention are possible in light of the above teachings. It is therefore to be understood that while the invention has been described in this specification with some particularity, it is not intended to limit the invention to the particular embodiments provided herein. On the contrary, it is intended to cover all alternatives, modifications, and equivalents as may be included within the spirit and scope of the invention.

All references cited herein are herein incorporated by reference in entirety.

We claim:

1. A liquid handling and detection system device for use with a planar binding reaction assay device, comprising:
(a) transportation means to movably locate said assay device through said system device;
(b) a liquid handling means for applying a sample to said assay device;
(c) a detection means for detecting presence or amount of a signal on said device;
(d) a data storage means, a data processing means, a data communication means or a combination thereof; and
(e) a housing of said system device.

2. The system device of claim 1, wherein said liquid handling means is automated to dispense plural samples.

3. The system device of claim 2, wherein said plural samples are dispensed simultaneously.

4. The system device of claim 2, wherein said plural samples are dispensed sequentially.

5. The system device of claim 1, comprising a data processing means.

6. The system device of claim 1, comprising a data storage means.

7. The system device of claim 1, comprising a data communication means.

8. The system device of claim 1, further comprising a data display means.

9. The system device of claim 1, wherein said planar assay device comprises an antibody.

10. The system device of claim 9, wherein said assay device comprises lateral flow.

11. The system device of claim 1, wherein said planar assay device comprises a length of sealable means for forming a sealed void therein, wherein said planar assay device is in said void.

12. The system device of claim 11, where said sealable means comprises two membranes.

13. The system device of claim 11, wherein said sealable means comprises a plurality of voids, each containing an assay device.

14. The system device of claim 1, wherein said assay device comprises a portable, removable container means which engages said housing.

15. The system device of claim 14, wherein said container means comprises a plurality of assay devices.

16. The system device of claim 12, wherein said membranes are sealable.

17. The system device of claim 12, wherein each said membranes comprises a collecting means.

18. The system device of claim 17, wherein said collecting means comprises a spool.
